(19) United States
(12) Patent Application Publication
(10) Pub. No.: US 2021/0169342 A1
(43) Pub. Date: Jun. 10, 2021

(54) Method and System for Estimating Pressure Difference in Turbulent Flow

(71) Applicant: King's College London, London (GB)

(72) Inventors: David NORDSLETTEN, London (GB); David MARLEVI, London (GB); Pablo LAMATA, London (GB)

(21) Appl. No.: 16/707,310

(22) Filed: Dec. 9, 2019

(51) Int. Cl. A61B 5/021; A61B 5/026

(52) U.S. Cl. CPC A61B 5/021; A61B 2562/0247; A61B 5/0263

(57) ABSTRACT

Aspects described herein estimate the pressure difference across a hollow region arising from fluid flow within the hollow region, based on an imaged fluid flow. The method utilises a complete description of fluid mechanical behaviour to derive an estimate of relative pressure or pressure difference over arbitrary flow segments. The method uses the concept of a virtual or arbitrary velocity field in the analysis of the work-energy of the fluid flow. Furthermore, the method uses statistical analysis to derive the acquired flow as a mean field and a related covariance quantity and uses this statistical description in the evaluation of virtual work-energy of the fluid flow. This assessment of virtual work-energy of the fluid flow is then used to derive an estimate of the pressure difference across any two given points (relative pressure) in the hollow region.

### FIELD

The present disclosure relates to a non-invasive method and a related system for estimating the relative pressure through a tubular segment, arising from fluid flow, more specifically turbulent fluid flow, in the tubular segment. In particular, it relates to a non-invasive method and a related system for estimating blood pressure drop through a blood vessel based on measurements obtained from medical imaging modalities which enable full-field acquisition of 3D flow.

### BACKGROUND

Flow abnormalities are typical indicators of cardiovascular disease (CVD). In the presence of valvular stenosis, the development of post-stenotic turbulence is directly related to pathological changes in cardiac workload (Schöbel et al., 1999; Dyverfeldt et al., 2013), and hemodynamic alterations in heart failure patients have been linked to pathological neurohormonal activation (Schrier and Abraham, 1999). With disease-related flow changes even proposed to occur prior to any detectable morphological change (Pedrizzetti et al., 2014), assessing hemodynamic behaviour is of direct clinical importance.

Several hemodynamic biomarkers have been proposed for the diagnosis of CVD. In particular, pressure drops or differences in relative pressure provide valuable clinical biomarkers for a range of CVDs. Transvalvular pressure drop is the recommended measure of stenotic severity (Baumgartner et al., 2009), and regional changes in relative pressure have been proposed as a determinant for interventional angioplasty (De Bruyne et al., 2006) or coarctated artery stenting (Jenkins and Ward, 1999). The pressure drop over the left ventricular outflow tract is also an established risk marker in hypertrophic cardiomyopathy patients (Bernard et al., 2011), and the clinical outcome for patients with pulmonary hypertension has even been linked to the degree of transpulmonary relative pressure (Galrn et al., 2015). Recent studies have also evaluated the production of turbulent flow in patients with aortic stenosis (Dyverfeldt et al., 2013; Bahlmann et al., 2010), indicating links between cardiovascular relative pressure and disease severity.

Current clinical assessment of pressure variations is largely based on Doppler echocardiography or intravascular catheterization. While catheterization is limited by its invasive nature (Wyman et al., 1988; Omran et al., 2003), in Doppler echocardiography 1D estimates of peak velocities are linked to pressure changes through the simplified Bernoulli equation (Stamm and Martin, 1983). Albeit effective for certain subsets of CVD, the simplification of the assessed fluid mechanical environment limits the method's general applicability. Extended and modified Bernoulli-based methods have been proposed (Garcia et al., 2000; Falahatpisheh et al., 2016), however discrepancies between Bernoulli estimates and invasive measures are still frequently reported (Baumgartner et al., 1999; Garcia et al., 2003; Donati et al., 2017; Feldman and Guerrero, 2016).

Recent advances in medical imaging have now enabled the full-field measurement of cardiovascular flow through 4D flow MRI (Dyverfeldt et al., 2015). Through refined sequencing, assessment of incoherent turbulent flow structures has also been achieved (Ha et al., 2017; Haraldsson et al., 2018). With this, a more comprehensive assessment of relative pressure is enabled where the Navier-Stokes equations—theoretically describing the flow of any viscous fluid—can be utilized in its complete form. Solution of a Pressure Poisson Equation (PPE) based on acquired 4D flow data has been proposed as a method for mapping relative pressure fields (Krittian et al., 2012; Bock et al., 2011); however, its accuracy depends on the defined flow domain and has shown bias when applied to stenotic flows in-silico (Donati et al., 2017; Bertoglio et al., 2018; Casas et al., 2016). Other methods have also been proposed, using a mixed PPE/Stokes approach (Svihlovà et al., 2016), or utilizing generated flow turbulence to quantify irreversible pressure changes. That turbulence can relate to increases in pressure has been extensively covered in both theoretical (Pope, 2001; Davidson, 2015) and clinical literature (Schöbel et al., 1999; Dyverfeldto et al., 2013), and originates from the fact that irregular velocity fluctuations will obstruct the natural passage of flow. Proposed turbulence-based methods have evaluated either turbulent kinetic energy alone (Dyverfeldt et al., 2015), incorporated an added shear-scaling approach (Gülan et al., 2017), or assessed turbulent energy dissipation directly from image data (Ha et al., 2017). Even though showing initial promise, the above methods have however all been limited to relatively simplified flow scenarios, and their applicability to transient turbulent flows remains somewhat unexplored.

Recently, the inventors derived a formulation that evaluates relative pressure using either a direct work-energy form of the Navier-Stokes equations (Work-Energy Relative Pressure, WERP (Donati et al., 2015)) as well as an alternative form where the work-energy of an auxiliary virtual field was evaluated (virtual Work Energy Relative Pressure, vWERP (Marlevi et al., 2019)). Utilizing vWERP, the inventors showed that accurate relative pressure estimates could be achieved in arbitrary multibranched vasculatures. Furthermore, vWERP was validated in-vivo against invasive catheterization. The cohort utilized was also such that alternative approaches were inherently obstructed by utilized spatiotemporal image resolution or challenging vascular anatomy. However, while promising, the proposed vWERP method did not include analysis of turbulent energy dissipation.

Given the above, there exists a need for a robust and an efficient method for analysing turbulent flow regimes in fluid flow, particularly turbulence-driven variations in blood flow in cardiovascular systems.

### SUMMARY OF DISCLOSURE

The present disclosure provides a non-invasive method to overcome the limitations associated with the above-mentioned prior art methods such as omissions of non-advective or turbulent energy components or limitations to single-vessel geometries.

One advantage of the techniques described herein is that it is non-invasive as it uses imaged fluid flow to estimate the pressure difference. Secondly, it has the advantage of enabling arbitrary probing of relative pressure throughout any imaged vascular structure while simultaneously accounting for turbulence-inducing high-flow regimes. This is because the method implements the concept of a virtual field to assess virtual work-energy elements of the fluid flow while also incorporating statistical analysis of the fluctuations in the fluid flow due to turbulence. In particular, when compared to the prior art as described above, the present method advantageously enables an accurate estimation of pressure difference across complex, tortuous multi-branched vascular structures for severely turbulent fluid flows. This is particularly beneficial when using relative pressure as a biomarker for cardiovascular diseases where turbulent flow is prevalent.

According to one or more aspects, a method is used to estimate the pressure difference across a hollow region arising from fluid flow within the hollow region, based on an imaged fluid flow. The method utilises a complete description of fluid mechanical behaviour to derive an estimate of relative pressure or pressure difference over arbitrary flow segments. The method uses the concept of a virtual or arbitrary velocity field in the analysis of the work-energy of the fluid flow. Furthermore, the method uses statistical analysis to derive the acquired flow as a mean field and a related covariance quantity and uses this statistical description in the evaluation of virtual work-energy of the fluid flow. This assessment of virtual work-energy of the fluid flow is then used to derive an estimate of the pressure difference across any two given points (relative pressure) in the hollow region.

According to a first aspect of this disclosure, there is provided a method of estimating pressure difference across a hollow region arising from fluid flow within the hollow region, the method comprising: acquiring a three-dimensional time-dependent image of the fluid flow; processing the acquired image to derive an expression of the mechanical behaviour of the fluid flow, wherein the expression comprises a component relating to stochastic flow fluctuations in the fluid flow; processing at least part of the derived expression of the fluid flow mechanical behaviour to define a fluid flow domain; computing an arbitrary, divergence-free velocity field (w) with null values on a lateral wall (Γ_{w}) of the fluid flow domain (Ω, Ω_{ROI}); processing components of the derived expression in combination with the arbitrary flow field to derive a work-energy expression for the fluid flow; and estimating the said pressure difference using elements of the derived work-energy expression.

According to a second aspect of this disclosure, there is provided a system comprising: a unit configured to receive imaged fluid flow data across a hollow region; a non-transitory computer-readable medium storing a program causing a computer to execute a process on the imaged fluid flow data for estimating a pressure difference due to the fluid flow across the hollow region, wherein the program is further adapted to cause the computer to estimate a virtual work-energy contribution to the pressure difference due to stochastic fluctuations in the fluid flow; a processor configured to execute the program stored on the non-transitory computer-readable medium; a unit configured to store the estimated pressure difference data.

According to a third aspect of this disclosure, there is provided a non-transitory computer-readable medium storing a program causing a computer to execute a process on imaged fluid flow data, the program comprising instructions to: acquire a three-dimensional time-dependent image of the fluid flow; process the acquired image to derive an expression of the mechanical behaviour of the fluid flow, wherein the expression comprises a component relating to stochastic flow fluctuations in the fluid flow; process at least part of the derived expression of the fluid flow mechanical behaviour to define a fluid flow domain; compute an arbitrary, divergence-free velocity field (w) with null values on a lateral wall (Γ_{w}) of the fluid flow domain (Ω, Ω_{ROI}); process components of the derived expression in combination with the arbitrary flow field to derive a work-energy expression for the fluid flow; and estimate the said pressure difference using elements of the derived work-energy expression.

According to another aspect of this disclosure, there is provided a method of determining a pressure difference across a tube arising from fluid flow within the tube, comprising: obtaining three-dimensional time-dependent fluid flow data; processing the three-dimensional time-dependent fluid flow data to acquire velocity field data (v) of the fluid flow; processing the obtained fluid flow data to define a fluid flow domain (Ω, Ω_{ROI}) over which the pressure difference is to be determined; computing a arbitrary velocity field (w), wherein the arbitrary velocity field is a solenoidal field with zero velocity on a lateral wall region (Γ_{w}) of the fluid flow domain (Ω_{ROI}); processing the obtained velocity field data (v) and the arbitrary velocity field (w) to determine virtual work energy components describing the fluid flow; and calculating the pressure difference in dependence on all of the virtual work energy components.

Further features of the disclosure are defined in the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of aspects described herein and the advantages thereof may be acquired by referring to the following description in consideration of the accompanying drawings, in which like reference numbers indicate like features, and wherein:

FIG. 1 is a flow chart describing a method according to one or more illustrative aspects described herein.

FIG. 2 is a drawing illustrating a method according to one or more illustrative aspects described herein.

FIG. 3 is a block diagram describing a system according to one or more illustrative aspects described herein.

FIGS. 4 to 9 are various graphs illustrating results and effectiveness of a method according to one or more illustrative aspects described herein.

### DETAILED DESCRIPTION

The present disclosure provides a method and a corresponding system for estimating the relative pressure across a hollow region arising from fluid flow in the hollow region. With relative pressure being a recognised clinical biomarker and with turbulent flow present in several cardiovascular diseases, the present disclosure addresses the problem of improving the accuracy of the assessment of relative pressure of fluid flow through complex, multibranched vascular segments, while still incorporating the effects of turbulent energy dissipation.

Starting from the work-energy principle, the derivation of the formula for pressure difference over a tubular segment arising from fluid flow within the hollow region, is first described. Subsequently, its discrete formulation and pre-processing steps required to work with imaged fluid flow data, in particular three-dimensional time-dependent fluid flow data, for example, 4D phase contrast magnetic resonance imaging (PC-MRI) data, is also described. Furthermore, results are presented which validate the present method in a set of in-vitro stenotic valve phantoms, and further evaluate method performance in a complex patient-specific time-dependent in-silico data set. In all cases, method performance is compared against alternative approaches, highlighting the benefits of the method of this disclosure as well as providing explanations to theoretical and practical differences between evaluated methods.

The derivation of the present method originates from the conservation of mass and momentum for an isothermal viscous Newtonian fluid, described by the Navier-Stokes equations as:

$$\begin{matrix}ρ \frac{\partial v}{\partial r} + ρ v \cdot ∇ v - μ {∇}^{2} v + ∇ p = 0 , & ( 1 ) \\ ∇ \cdot v = 0. & ( 2 )\end{matrix}$$

where v is the velocity field, ρ the pressure, p fluid density, and μ dynamic viscosity of the fluid.

In the present method, it is assumed that the assessed flow behaviour has a quasi-period T, meaning that the flow field exhibits semi-periodic flow behaviour (i.e. v(t)˜v(t+iT) for any integer i∈N) except within incoherent turbulent flow regions where more significant variability exists. Supposing that the data is collected over n cycles, we can then define a linear expected value operator [symbol] [⋅] such that

$$𝔼 [ f ( t ) ] := \frac{1}{n} {\sum }_{t = 1}^{t} f ( t + kT ) , t ∈ [ 0 , T ] \qquad ( 3 )$$

for any given function ƒ:[0,nT]→ [symbol] ^{m}, (m=1, 2, 3). Following from the linearity of [symbol] , along with its commutativity with spatiotemporal derivative operators, applying [symbol] on equations 1-2 gives

$$\begin{matrix}ρ \frac{\partial [ v ]}{\partial i} + ρ ∇ \cdot 𝔼 [ vv ] - μ {∇}^{2} 𝔼 [ v ] + ∇ 𝔼 [ p ] = 0 , & ( 4 ) \\ ∇ \cdot 𝔼 [ v ] = 0. & ( 5 )\end{matrix}$$

Assigning V= [symbol] [v] and P= [symbol] [p] to be the acquired mean field quantities, equations 4-5 can be re-expressed as:

$$\begin{matrix}ρ \frac{\partial V}{\partial t} + ρ v \cdot 𝔼 [ vv ] - μ {∇}^{2} V + ∇ P = 0 , & ( 6 ) \\ ∇ \cdot V = 0. & ( 7 )\end{matrix}$$

Following the functionality of [symbol] , it can be shown that

[symbol]

[vv]=VV+Cov[v,v], (8)

where Cov[v, v]= [symbol] [(v−V)(v−V)] is the covariance of the observed flow. Utilising this, a final form of the Navier-Stokes equations including incoherent flow regimes is derived as follows:

$$\begin{matrix}ρ \frac{\partial V}{\partial t} + ρ ∇ \cdot ( VV ) + ρ ∇ \cdot Cov [ v , v ] - μ {∇}^{2} V + ∇ P = 0 , & ( 9 ) \\ ∇ \cdot V = 0. & ( 10 )\end{matrix}$$

Derivation of the virtual work-energy equation can then be achieved by multiplying equation 9 by an arbitrary velocity field w, and integrating over the entire fluid domain Q with boundary r and normal n. However, when incorporating incoherent flow behaviour, an additional turbulent energy dissipation term Tₑ appears following these stochastic fluctuations, rendering a complete energy balance reading:

$$\begin{matrix}\frac{\partial {K}_{e}}{\partial t} + {A}_{e} - {S}_{e} + {V}_{e} + H ( p ) + {T}_{e} = 0 , & ( 11 ) \\ with & \\ \frac{\partial {K}_{e}}{\partial t} = {\int }_{Ω} ρ \frac{\partial V}{\partial t} \cdot w d Ω & ( 12 ) \\ {A}_{e} = {\int }_{Ω} \vert ρ ∇ \cdot ( VV ) ) \cdot w \vert d Ω & ( 13 ) \\ {S}_{e} = {\int }_{Γ} ( μ ∇ V \cdot n ) \cdot wd Γ & ( 14 ) \\ {V}_{e} = {\int }_{Ω} μ ∇ V : ∇ wd Ω & ( 15 ) \\ H ( p ) = {\int }_{Γ} pw \cdot nd Γ - {\int }_{Ω} p ∇ \cdot wd Ω & ( 16 ) \\ {T}_{e} = {\int }_{Ω} ρ ( ∇ \cdot Cov [ v , v ] ) \cdot wd Ω & ( 17 )\end{matrix}$$

Each entity in equation 12-17 represents a different virtual energy component in the assessed system. Most intuitively understood is the case where w=v, in which the energy components correspond to real work-energy: Kₑ representing kinetic energy held within the fluid, Aₑ the rate at which kinetic energy enters, exits, or changes within Ω, Sₑ the power transfer into the system due to shear, Vₑ the rate of viscous energy dissipation, H(p) the input hydraulic power, and Tₑ the turbulent energy dissipation.

With the above, the relative pressure between two arbitrary boundaries can be isolated by assigning certain attributes to w. Specifically, by splitting the domain boundary into an inlet, outlet and wall domain, respectively (Γ=Γᵢ∪Γ [symbol] ∪Γ_{w}), and by choosing w to be a solenoidal field (Λ·w=0) assigned as w=0 at Γ_{w}, H(p) can be expressed as:

$$H ( p ) = {\int }_{Γ} pw \cdot ndΓ = \text{?} pw \cdot nd Γ , \\ \text{?} \text{indicates text missing or illegible when filed} \qquad ( 18 )$$

which, under the condition of mass conservation of w (that is, the total inflow Q through Γᵢ has to be identical to the outflow through Γₒ), simplifies into

$$H ( p ) = p {\int }_{{Γ}_{1}} w \cdot nd Γ + {p}_{a} {\int }_{{Γ}_{a}} w \cdot nd Γ = Δ pQ , \qquad ( 19 )$$

with pₖ being the w·n—weighted mean pressure on boundary k, and Δp being the relative pressure between Γₗ and Γₐ.

Further, by letting w act in the surface normal of Γ and knowing that the spatial gradients of v are negligibly small at the vicinity of the surface boundary, the shear input term Sₑ becomes effectively negligible.

Lastly, again making use of the fact that w=0 at Γ_{w} and assuming that the chosen Γᵢ and Γₒ are sufficiently far away from the core turbulence regions (that is, Cov[v, v]≈0 at Γ [symbol] ), Tₑ can be simplified into:

$$\begin{matrix}{T}_{e} = {\int }_{Ω} ρ ( ∇ \cdot Cov [ v , v ] ) \cdot wd Ω & ( 20 ) \\ = {\int }_{Ω} ρ ( Cov [ v , v ] n ) - wd Γ - {\int }_{Ω} ρ Cov [ v , v ] : ∇ wd Ω & ( 21 ) \\ = {\int }_{{Γ}_{1} ⋃ {Γ}_{2}} ρ ( Cov [ v , v ] n ) \cdot wd Γ - {\int }_{Ω} ρ Cov [ v , v ] : ∇ wd Ω & ( 22 ) \\ {\int }_{Ω} ρ Cov [ v , v ] : ∇ wd Ω . & ( 23 )\end{matrix}$$

Summarizing all of the above, we end up with a final isolated expression of Δp, specifically given as

$$Δ p = - \frac{1}{Q} ( \frac{\partial }{\partial t} {K}_{e} + {A}_{e} + {V}_{e} + {T}_{e} ) , \qquad ( 24 )$$

with all the right-hand side terms directly derivable from the acquired flow field.

The final step of the present method is the identification of a virtual or arbitrary velocity field w abiding to all of the aforementioned assumptions. The arbitrary velocity field (w) can be computed as any virtual field that fulfils the boundary conditions as specified in the derivation of the method above—in particular, the arbitrary velocity field is assigned to be a solenoidal field (∇·w=0) with zero velocity on a lateral wall region (Γ_{w}) of the fluid flow domain. In one embodiment, w is solved as a solution to a Stokes problem specifically defined as

$$\begin{matrix}{∇}^{2} w + ∇ λ = 0 & ( 25 ) \\ ∇ \cdot w = 0 & ( 26 ) \\ w = \{ \begin{matrix}- n ( 1 - {( \frac{r}{R} )}^{2} ) , & Γ , \\ 0 , & {Γ}_{w}\end{matrix} & ( 27 )\end{matrix}$$

where λ is the virtual pressure field corresponding to the arbitrary velocity field (w) and n is the normal vector on an inlet plane Γᵢ of a boundary r of the flow domain Ω, where a parabolic inflow is defined at Γᵢ in a direction of the normal n, at a radial position r with a perimeter radius R. It is also noted that there are no constraints defined for w at the outlet plane Γₒ.

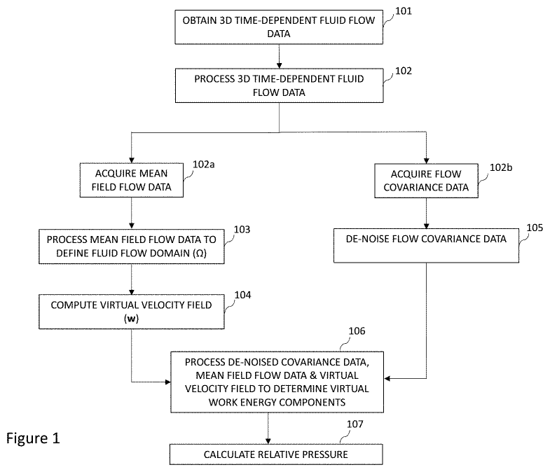

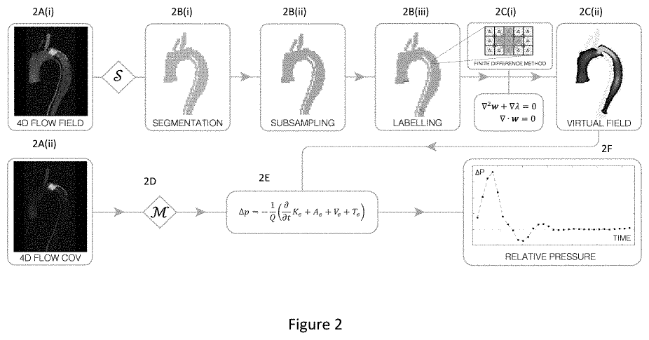

An embodiment relating to the numerical implementation of the above-presented method onto acquired three-dimensional time-dependent fluid flow data will now be described with reference to the flow chart in FIG. 1 and the corresponding illustrative overview of the process in FIG. 2.

FIG. 1 indicates that the first step in the numerical implementation of the method is obtaining three-dimensional time-dependent fluid flow data (step 101) which, in one embodiment may be 4D flow data obtained using 4D PC-MRI. This is followed by processing the obtained three-dimensional time-dependent fluid flow data to derive mean field quantities (step 102a, FIG. 2A(i)) and corresponding flow covariance (step 102b, FIG. 2A(ii)). The mean field data is processed to define a fluid flow domain (Ω, Ω_{ROI}) over which the pressure difference is to be determined (step 103, FIG. 2B(i)-(iii)). This can include creating a segmented (S) or voxelised fluid flow domain (FIG. 2B(i)), followed by domain subsampling and labelling (step FIGS. 2B(ii) and 2B(iii)). Then, a arbitrary velocity field (w), which, in one embodiment, is a Stokes flow virtual field, is computed by means of a finite difference method scheme (step 104, FIGS. 2C(i) and 2C(ii)). In parallel, the acquired covariance data is masked (M) to remove non-physical negative diagonal entries (step 105, FIG. 2D). The data is then processed to determine virtual work energy components (step 106, FIG. 2E), deriving relative pressure by means of virtual work-energy assessment (step 107, FIG. 2F). In this way, relative pressure between the selected inlet and outlet planes is computed as a function of time.

Steps 103-105 in the above method, which also describe the process required for computing a arbitrary velocity field w, will now be described in more detail with reference to FIG. 2.

In relation to creating a segmented fluid flow domain, the segmentation can be performed by the creation of a binary mask and thresholding on generated velocity magnitude images, separating fluid domain voxels from neighbouring static background (FIG. 2B(i)).

The segmented fluid flow domain may be further processed to remove noise particularly in cases where the obtained data is not pre-processed or is not simulated data. Spatial noise filtering can be performed, preferably using a k-order 3D polynomial fitting to approximate the signal within a defined voxel neighbourhood, with optimal filter settings (interpolation order and kernel size) defined by translating the estimated data noise level to a simplified 1D sinusoidal signal.

Next, inlet and outlet planes between which pressure drops are to be calculated, are selected. In one embodiment, these planes are manually selected within the segmented flow field (FIG. 2B(iii)). Specifically, plane positions may be manually initiated before automatic adjustments are applied to assign the inlet/outlet planes as perpendicular cross-sections to the region of interest. Labelling of the segmented domain is also performed. This is preferably performed by initiating voxel-wise region growing from the defined inlet plane, with all voxels inside the assessed domain labelled as: interior (entirely within the fluid domain), exterior (entirely outside the fluid domain), inlet/outlet (inside defined inlet/outlet planes), or wall (separating interior and exterior), respectively. Labelling of the segmented domain in this way helps to appropriately assign boundary conditions (refer derivation of the method above for boundary conditions) in the arbitrary velocity field computation, as will be described below.

Prior to the computation of arbitrary velocity field (w) computation, in some embodiments, in order to improve the numerical accuracy of this computation, domain subsampling may be performed as part of the proposed method. Specifically, the input data base-discretisation is subdivided by a chosen integer value, converting a single image voxel into several uniformily distributed nodal points. Domain subsampling advantageously improves the accuracy of w and is complemented by an additional subsampling of v. In one embodiment, domain subsampling is employed by a factor of 2, subdividing a single image voxel into 8 uniformily distributed subvoxels, each inheriting the value of the original parent voxel.

Using the labelled fluid domain above, the virtual field is computed. As mentioned earlier in the derivation of the method above, the arbitrary velocity field (w) can be computed as any virtual field that fulfils the boundary conditions of a solenoidal field (∇·w=0) with zero velocity on a lateral wall region (Γ_{w}) of the fluid flow domain (see equation 27 above). Preferably, w is solved as a solution to a Stokes problem using a staggered-grid Finite Difference Method (FDM) approach where the boundary conditions specified in equation 27 above are projected onto the discretized labelled domain. In one embodiment, an iterative solver based on the BFBT preconditioning method (Elman et al., 2006) is used to solve the FDM liner equation system.

For spatiotemporally discretised flow field data, the masked flow covariance data, the acquired mean field flow data and the computed arbitrary velocity field data are combined as below to re-express the equation 24 for estimating pressure difference as:

$$Δ {p}_{t + \frac{1}{2}} = - \frac{1}{Q ( w )} ( \frac{\partial }{\partial t} {K}_{e} ( {v}_{t + \frac{1}{2}} , w ) + {A}_{e} ( {v}_{t + \frac{1}{2}} , w ) + {V}_{e} ( {v}_{t + \frac{1}{2}} , w ) + {T}_{e} ( {Cov}_{t + \frac{1}{2}} , w ) , \qquad Eq . 28$$

with

$${v}_{t + \frac{1}{2}}$$

derived as the mean of vₜ and vₜ₊₁. A similar mean is derived for

$${Cov}_{t + \frac{1}{2}} .$$

With the above, each separate energy component in equation 24 are calculated by integration over voxelised version of Ω and Γᵢ and Γₒ, here referred to as Ω_{ROI}, Γ_{I,ROI} and Γ_{O,ROI}, respectively. With an image voxel identified by its indices (i, j, k), the energy terms can then be computed as

$${K}_{e} ( v , w ) = ρ dV \text{?} ( v ( i , j , k ) \cdot w ( i , j , k ) ) \\ {A}_{e} ( v , w ) = ρ dV \text{?} ( \vert v ( i , j , k ) \cdot G ( v ) ( i , j ) \vert \cdot w ( i , j , k ) ) \\ {V}_{e} ( v , w ) = ρ dV \text{?} ( G ( v ) ( i , j , k ) : G ( w ) ( i , j , k ) ) \\ {T}_{e} ( v , w ) = - ρ dV \text{?} ( Cov [ v , v ] ( i , j , k ) : G ( w ) ( i , j , k ) ) \\ Q ( w ) = dS \text{?} ( w ( i , j ) \cdot N ( i , j ) ) , \\ \text{?} \text{indicates text missing or illegible when filed} \qquad ( Eq . 29 \text{-} Eq .33 )$$

where the inlet plane (Γ_{I,ROI}) and the outlet plane (Γ_{O,ROI}) of the fluid domain (Ω_{ROI}) have corresponding normal vectors (N) and where (i, j, k) are voxel indices; dS=Π [symbol] ²Δxᵢ and dV=Π [symbol] ³Δxᵢ are the pixel area and voxel volume, respectively, both based on the voxel length Δxᵢ in each spatial dimension; and G(⋅) is the discretized gradient operator, defined as a spatial central-difference operator with one-directional derivatives employed at boundaries of the fluid flow domain.

One step of the method according to one or more aspects is the de-noising of the flow covariance data (step 105, FIG. 2D). In comparison to noise filtering of the mean field quantities (as described above in relation to creating the segmented fluid flow domain), application of the same to covariance data (Cov[v, v]) is less trivial. For de-noising the flow covariance data, in order to avoid non-physical entries, voxels where negative diagonal entries appeared are masked away from the analysis (that is, keeping strictly positive fluctuations of Cov[vₓ, vₓ], Cov[v_{y}, v_{y}], and Cov[v_{z}, v_{z}], respectively).

In addition to the details of the method mentioned above, if using 4D flow MRI data, a vital pre-processing step lies in the calibration and correction of potentially erroneous data, following the presence of, for example, eddy currents, field inhomogeneities, or regions of signal aliasing.

According to an aspect, a method for estimating pressure difference across a tubular segment is not bound to any specific imaging modality and can be implemented on any type of system which enables full-field acquisition of 3D flow.

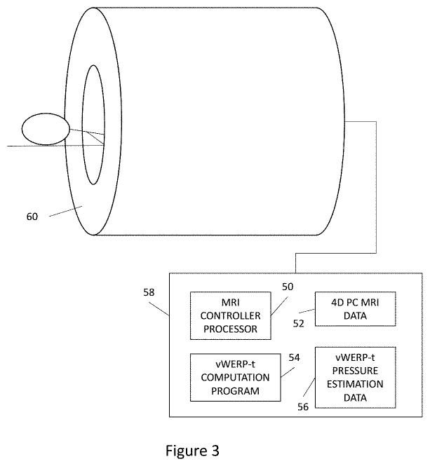

FIG. 3 illustrates an embodiment of an imaging system that is able to apply the above described techniques to calculate blood flow through a blood vessel using the above-described method which is also named by the inventors as “vWERP-t”—that is, virtual Work Energy Relative Pressure method accounting for turbulent flow.

In FIG. 3, a 4D phase contrast magnetic resonance imaging (4D PC MRI) system 60 is provided, comprising MR imaging coils within which the subject is located, controlled by an MRI imaging control system 58, including an MRI controller processor 50. The MRI imaging control system 58 including the MRI controller processor 50 function in a conventional manner to allow 4 dimensional phase contrast magnetic resonance image data to be obtained, for example of an internal blood vessel of the subject the pressure difference across it is desired to know. For example, it may be desirable to measure transvalvular pressure drops (TPD) along the transvalvular region in the heart, between the left ventricular outflow tract and the vena contracta. Of course, other blood vessels may also be monitored, as desired. For example, it may be possible to take into account secondary branches.

The 4D PC MRI system 58, 60, collects 4D PC MRI data 52 of the imaging subject, which is saved for further processing and analysis. It is noted that the complete velocity vector field over the entire imaged plane, and not simply its projection along the insonation line, is available from 4D PC-MRI.

The 4D PC MRI system 58, 60, also includes a vWERP-t computation program 54, which acts to process the 4D PC MRI data 52 as described above in accordance with the vWERP-t processing method described, to obtain vWERP-t pressure estimation data 56. The vWERP-t processing method computes the pressure difference along the vessel as an addition of four virtual rates of energy transfer (kinetic, advected, viscous and turbulent energy dissipation rates) divided by the net flow through the vessel, as described above. The resulting vWERP-t pressure estimation data 56 gives an estimate of pressure at each point along the vessel between the measured inlet and outlet planes, taking into effect the vessel walls between the inlet and outlet. It provides a complete solution for every point within the vessel between the inlet and outlet.

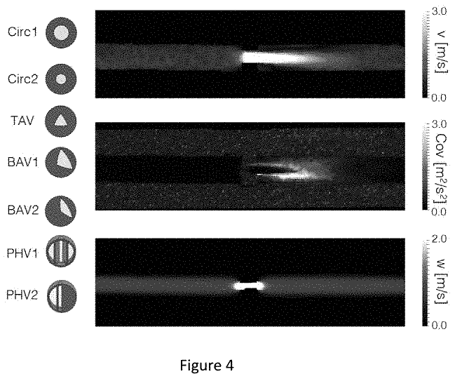

A comparative evaluation of the present method (which is henceforth referred to as vWERP-t) with some other energy-based methods for relative pressure estimation of turbulent flows, will be discussed below in relation to FIGS. 4-8.

Firstly, vWERP-t is compared with a method which the inventors refer to as “vWERP” which uses the concept of arbitrary velocity field (w) but with no consideration of turbulent or incoherent flow fluctuations. Therefore, in vWERP formulation non-invasive relative pressures are derived by:

$$Δ p = - \frac{1}{Q} ( \frac{\partial }{\partial t} {K}_{e} + {A}_{e} + {V}_{e} ) , \qquad ( 34 )$$

excluding the turbulence-induced Tₑ term given in equation 24. In cases of significant turbulent energy dissipation, vWERP is thus expected to diverge from vWERP-t.

Secondly, an extended real work-energy formulation WERP-t can be computed by assigning w=v within equations 12-17. With such, WERP-t is identical to equation 24, however with the energy components reflecting the real work-energy of the acquired flow field. Utilizing a real work-energy approach has the advantage of not having to compute a virtual field. However, when w=v, the real flow Q in equation 24 will now be a function of the acquired field, causing potential divergent behaviour during late diastolic phases (where Q→0), as well as in branching vasculatures (where, Q|Γᵢ≠Q|Γₒ violating assumptions in the derivation of equation 19).

Thirdly, vWERP-t is compared to another method, here denoted as Turbulence Production or TP method (refer Ha et al., 2017, 2019 for details on TP method). TP evaluates the total real work-energy within the assessed fluid domain. In the TP method, flow covariance data is obtained but is masked to remove all negative integral arguments for any velocity direction and any voxel (i, j, k) as shown below:

$$- Cov [ v , v ] \text{:} ∇ V = {\sum }_{α = 1}^{3} {\sum }_{β = 1}^{3} max ( - Cov [ {v}_{α} , {v}_{β} ] \frac{\partial {V}_{α}}{\partial {X}_{β}} , 0 ) \qquad ( Eq . 35 )$$

It should however be pointed out that there exists no theoretical reason justifying the conservative masking in Equation 35, and in principle negative entries can appear at least for the off-diagonal covariance terms (i.e. for Cov[vᵢ, vⱼ] where i≠j).

One drawback of the TP method is that its performance, unlike that of the described vWERP-t method, is potentially affected by branching or diastolic flows. Furthermore, as will be shown below, a comparison of results of the described vWERP-t method with the TP method show that the constricting masking of TP does seem to skew results in cases where negative turbulence components are required to accurately capture relative pressure behaviour. Instead, vWERP-t represents a viable option where contributions from kinetic, advected, viscous and turbulent energy components together enable accurate assessment of relative pressure.

An outline of the tests performed to verify and validate vWERP-t is presented below.

Validation in a Steady-State Turbulent Flow

The vWERP-t approach, along with other benchmark methods were evaluated on a series of steady-state turbulent flow cases. This was done to isolate the turbulence-driven relative pressure from transient flow behaviours, otherwise shown to impact vascular pressure drops in-vivo (Donati et al., 2015; Lamata et al., 2014).

A previously published data set was utilized (Ha et al., 2019) where turbulent flow through seven 3D-printed aortic valve phantoms had been imaged by 4D flow MRI with six-directional icosahedral flow encoding (ICOSA6). Imaging was performed at 1.5 T (Philips Achieva, Philips Medical Systems, Best, the Netherlands), with velocity encoding: 1-5 m/s, spatial resolution: 1.5 mm³, and number of signal averages: 5. For each valve, imaging had been performed at four different flow rates, with Reynolds numbers ranging from ˜5552 to ˜20′000 in each case. Additionally, invasive pressure measurements were obtained for all valves and flow rates using installed pressure ports inside the custom-made flow circuit. For each acquisition, data velocity offset was corrected for by subtracting a static background image, and median filtering was employed to smooth the acquired velocity field. Mean field flow quantities and incoherent turbulence-driven flow fluctuations were acquired for all phantoms and flow rates, respectively.

A visualization of the seven valve configurations, along with an example of the acquired mean field, covariance, and corresponding virtual field are given in FIG. 4.

Validation in a Transient Turbulent Flow

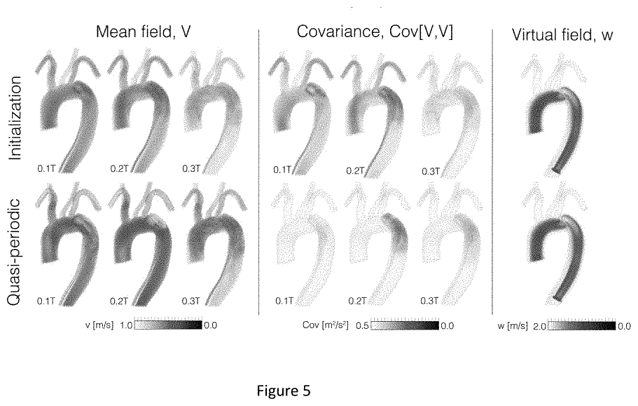

Expanding from the steady-state stenotic phantoms, evaluations of a transient flow case was also performed. Specifically, a patient-specific in-silico model of an acute type B aortic dissection (AAD) was utilized, for which 4D flow and pressure was generated using Computational Fluid Dynamics (CFD) simulations with a stabilized variational approximation of the Navier-Stokes equation for laminar and turbulent flows (Whiting and Jansen, 2001). With model and simulations details provided elsewhere (Dillon-Murphy et al., 2016), data from 10 consecutive simulations cycles were generated for the specific case of turbulent energy analysis. The simulated data set was then projected onto a uniform grid of 2 mm³, with data sampled at 32 time slices/cardiac cycle. From the sampled simulation data, mean field and covariance quantities were generated in two separate sets: one derived from the first 3 simulated cardiac cycles, representing an initiation phase where flow field covariance was enhanced following large inter-cycle variations, and one derived from the last 7 simulated cardiac cycles, where a more quasi-periodic flow behaviour was observed with subsequent lower covariance. For both sets of mean field and covariance data, relative pressures were estimated from the left ventricular outflow tract, down to an approximate 180° bend of the descending false lumen of the thoracic aorta. Estimations were performed using vWERP-t along with other methods as discussed above. A visualization of mean field, covariance, and virtual field for the two different phases is provided in FIG. 5.

Statistical Analysis

Linear regression was assessed to quantify the relationship between predicted and true pressure drop in the in-vitro stenotic flow phantoms. Additionally, Bland-Altman plots were generated to assess potential method bias.

For the transient AAD-case, again linear regression analysis and Bland-Altman plots were generated with each time frame considered a separate data point. Additionally, the mean error was evaluated as

$${ɛ}_{Δ p} = ( \frac{\sqrt{{\sum }_{n = 1}^{N - 1} {( Δ {p}_{e} \text{?} - Δ p \text{?} )}^{2}}}{\sqrt{{\sum }_{n = 1}^{N - 1} Δ p \text{?}}} ) , \\ \text{?} \text{indicates text missing or illegible when filed} \qquad ( Eq . 36 )$$

where

$$Δ {p}_{e} \text{?}$$

$$\text{?} \text{indicates text missing or illegible when filed}$$

is the estimated Δp at discrete time step

$$\text{?}$$

$$\text{?} \text{indicates text missing or illegible when filed}$$

(representing the mean of tₙ and tₙ₊₁ as per equation 28), and

$$Δ p \text{?}$$

$$\text{?} \text{indicates text missing or illegible when filed}$$

is the corresponding true data. N is the number of temporal sample points (N=32).

For all of the above, the data analysis and method implementation was performed using MATLAB R2016a (MathWorks, Natick, Mass., USA).

Results

Results for the above-mentioned analyses relating validation of the vWERP-t approach, along with other benchmark methods, for steady-state turbulent flow and transient turbulent flow are presented below.

Steady-State Turbulent Flow

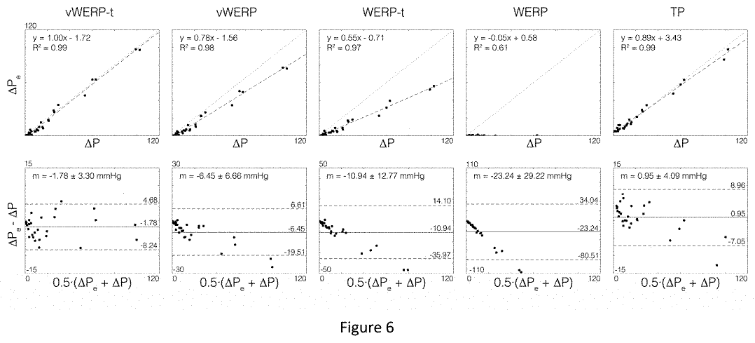

Linear regression and Bland-Altman plots for each estimation method are presented in FIG. 6. Over all acquisitions, vWERP-t showed a mean error of −1.8±3.3 mmHg, with a linear regression slope of k=1.00. The non-extended vWERP showed a mean error of −6.5±6.7 mmHg, with a linear regression slope of k=0.78. For the real work-energy approaches, TP showed a mean error of 1.0±4.1 mmHg, with a linear regression slope of k=0.89, whereas comparably larger errors were observed for WERP-t and WERP, with mean errors of −10.9±12.8 mmHg, and −23.2±29.2 mmHg, respectively, and linear regression slopes of k=0.55 and k=−0.1, respectively.

For the virtual vWERP-t approach, Aₑ contributed to 76% of the total relative pressure, with 21 and 3% coming from Tₑ and Vₑ, respectively. For the real WERP-t scenario, Tₑ accounted for 93% of the total relative pressure, with 11 and 2% coming from Vₑ and Aₑ, respectively. Note that no contribution was given from the kinetic [symbol] Kₑ, following the steady-state nature of the performed experimental mean flow.

Transient Turbulent Flow

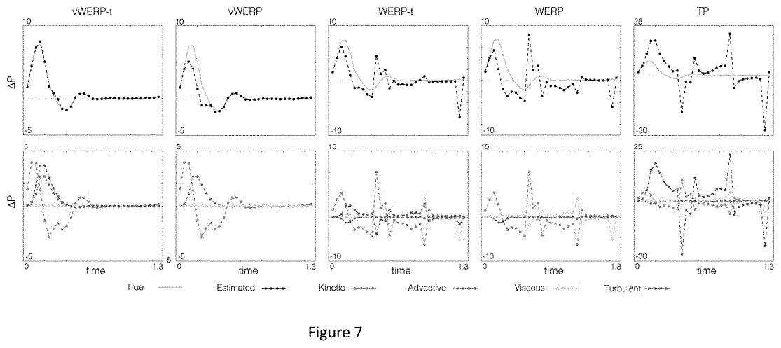

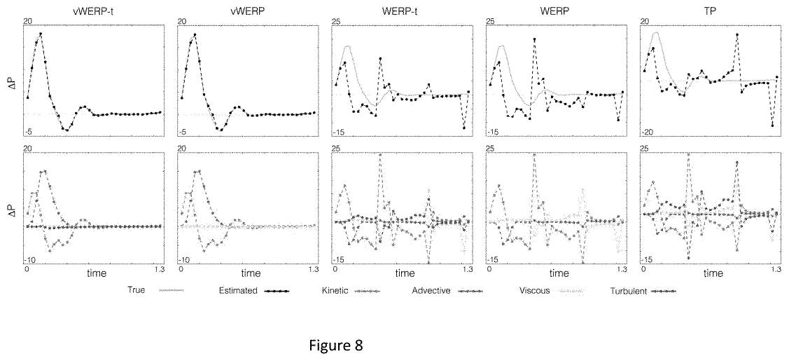

Estimations of relative pressure through the patient-specific AAD as a function of the different estimation approaches are given in FIGS. 7-8 for the initialization and quasi-periodic phase, respectively. Additionally, each subfigure is provided together with a detailing plot showing the variation of different virtual energy components as a function of time.

For vWERP-t, a mean error of 6.8% was given for the initialization phase, with the error changing to 6.2% during the quasi-periodic stabilization. For the non-extended vWERP, the corresponding mean errors were 47.8% and 6.9%. Regarding the real work-energy approaches WERP-t, WERP, and TP, mean errors of 89.0%, 126.0% and 362% were seen during the initialization phase, with values changing to 115.2%, 136.1% and 115.4% during the quasi-periodic stabilization.

For vWERP-t during initialization, the relative pressure at peak systole consisted of 18.5%

$$\frac{\text{?}}{\text{?}} {K}_{e} , \\ \text{?} \text{indicates text missing or illegible when filed}$$

34.3% Aₑ, 0.3% Vₑ and 46.9% Tₑ. During quasi-periodic stabilization, the same components changed to 17.6%

$$\frac{\text{?}}{\text{?}} {K}_{e} , \\ \text{?} \text{indicates text missing or illegible when filed}$$

82.6% Aₑ, 0.3% Vₑ and 0.5% Tₑ. For the real-work WERP-t, the relative pressure at peak systole during initialization consisted of 43.1%

$$\frac{\text{?}}{\text{?}} {K}_{e} , \\ \text{?} \text{indicates text missing or illegible when filed}$$

18.4% Aₑ, 3.8% Vₑ and 34.8% Tₑ, whereas the same entities changed to 43.4%

$$\frac{\text{?}}{\text{?}} {K}_{e} , \\ \text{?} \text{indicates text missing or illegible when filed}$$

48.8% Aₑ, 4.2% Vₑ and 3.6% Tₑ during quasi-periodic stabilization.

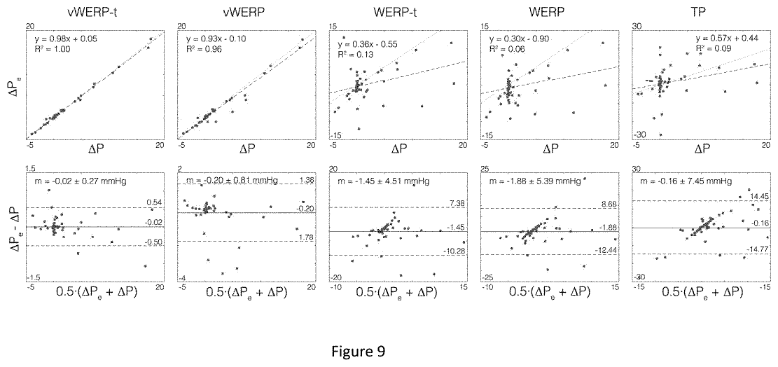

For a cumulative summation of the above estimated traces, FIG. 9 shows linear correlation and Bland-Altman plots for the in-silico estimations. Over both evaluated phases, and for all discrete time points, vWERP-t showed a mean error of 0±0.3 mmHg, with a linear regression slope of k=1.00. The non-extended vWERP has a mean error of −0.2±0.8 mmHg, with a linear regression slope of k=0.93. For the real work-energy approaches WERP-t, WERP, and TP, mean errors of −1.5±4.5 mmHg, −1.9±5.4 mmHg, and, −0.1±7.5 mmHg, respectively. Corresponding linear regression slopes were k=0.36, 0.30, and 0.57, respectively.

Discussion of Results

The significance of the above results is that vWERP-t has been shown to compare favourably against other relative pressure estimation methods—especially when applied on realistic, transient cardiovascular flows—the uniqueness of the present method is highlighted. The significance of the above results will be discussed in more detail below.

In the comparative study as detailed above, the inventors have presented a virtual work-energy method, vWERP-t, incorporating turbulent energy dissipation to accurately assess cardiovascular relative pressure in potentially turbulent flow directly from acquired velocity data. By including stochastic flow fluctuations in the theoretical derivation, the inventors have demonstrated that accurate estimates of relative pressure can be achieved both in-silico and in-vitro.

Accuracy and Comparative Performance of vWERP-t in Steady-State Turbulent Flows

For the evaluated in-vitro stenotic flow phantoms, vWERP-t showed excellent accuracy, showcasing a practically 1:1 relation against reference measures (k=1.00, R²=0.98). The inventors have observed that deviations were slightly larger in valves with lower flow magnitudes, with the prosthetic heart valve with one blocked leaflet (PVH1) having a vWERP-t mean error of 47%, corresponding to an absolute error of −3.1 mmHg. Comparably, the tricuspid aortic valve (TAV)—the valve phantom experiencing highest relative pressure—had a vWERP-t mean error of 14% or 2.2 mmHg. As shown in the Bland-Altman plots in FIG. 6, however, only a slight underestimation of −0.9±3.3 mmHg was seen over all phantoms, again highlighting the accuracy of the method extension described herein.

The importance of incorporating turbulent energy dissipation when assessing turbulent flow fields is also highlighted when comparing against results from the vWERP approach. Here, a systematic underestimation is apparent, with both decreased linear regression (k=0.78, R²=0.89), and increased mean error (−6.45±6.7 mmHg). Hence, using the virtual work-energy approach, in average 21% of the derived relative pressure is accounted for by turbulent energy dissipation, and is consequently required to accurately recover true relative pressure.

Comparing against real work-energy approaches, slightly different results were obtained for WERP-t, WERP, and TP. For WERP-t, again a systematic underestimation was observed, with increasing absolute errors at increasing relative pressure (k=0.55, R²=0.97, mean error of −10.9±12.8 mmHg). Again the valve phantoms with lower flow magnitudes experienced highest relative errors of 56 and 71% for PVH1 and the second prosthetic valve phantom, PVH2, respectively, equaling an absolute error of −3.4 and −5.7 mmHg. Conversely, the high flow magnitude TAV showed a mean relative error of 35%, equaling an absolute error of −20 mmHg. Noteworthy is that for the real work-energy approaches, the relative pressure was almost exclusively governed by turbulent energy dissipation, contributing to in average 93% of the entire relative pressure. This is also the reason to why real work-energy WERP, which does not account for turbulence, shows significant output deterioration, with barely any of the valve phantoms assessed accurately. The TP method rendered deviations from a 1:1 correlation observed (k=0.89, R²=0.98) (refer Ha et al., 2019).

For other alternative estimation approaches not relying on work-energy estimations from the complete Navier-Stokes equations (such as simplified and extended Bernoulli, or shear scaling methods), associated method simplifications were reflected by deterioration in estimation accuracy.

Accuracy and Comparative Performance of vWERP-t in Transient Turbulent Flows

The patient-specific in-silico data set was utilized as an extension of the detailed steady-state in-vitro analysis. The simulated data output did not only allow for controlled assessment of realistic flow behaviour, but did also serve as a clinically relevant example where turbulent energy dissipation act together with advective, kinetic, and viscous energy components in contributing to the total work-energy of the assessed vasculature.

As reported, vWERP-t showed high accuracy (cumulative linear regression of k=0.98, R²=1.00), with the relative pressure traces accurately captured both during phases of significant turbulent energy dissipation (initialization) as well as during phases where other energy components dominated relative pressure behaviour (quasi-periodic stabilization). Comparing to the vWERP results, the importance of including turbulent energy dissipation is again underlined, where the mean error increased from 6.8% to 47.8% when disregarding Tₑ in the virtual work-energy evaluation (i.e. going from vWERP-t to vWERP). The deviation was particularly evident at peak systole, again highlighting the clinical relevance of vWERP-t, where peak systolic metrics are typically the ones used to rep-resent the hemodynamic quantification in clinics.

Comparing against real work-energy approaches, a distinct reduction in accuracy was observed over all evaluated methods. For WERP-t and WERP, mean errors of above 89.0% were seen during both the initialization and quasi-periodic phase, and as evident in FIG. 7-8, severe output deterioration is observed during the diastolic phase, with method divergence following as the real flow Q→0 (comments on virtual versus real work-energy behaviour is discussed later in the description below). TP had similar problems during diastole, but did also present a pronounced overestimation of peak systolic relative pressure during the high-turbulent initiation phase. As seen in the inlet plot on energy components in FIG. 7-8, the TP overestimation comes from a dominant Tₑ, where the omission of negative turbulence production causes an overshoot in retrieved turbulent energy dissipation. Thus, the constricting masking of TP does seem to skew results in cases where negative turbulence components are required to accurately capture relative pressure behaviour. Instead, vWERP-t represents a viable option where contributions from kinetic, advective, viscous, and turbulent energy components together enable accurate assessment of relative pressure.

Comparative Performance Between Virtual and Real Work-Energy Approaches

The inventors have demonstrated that vWERP-t has an improved performance over real work energy approaches, for example WERP-t or TP.

The benefit of vWERP-t is particularly worth highlighting in conjunction to the real work-energy equivalent of WERP-t. In particular, real work-energy approaches will be obstructed by branching flows, where the real flow Q through the defined inlet plane Γᵢ is not equivalent to the real flow through the defined outlet plane Γₒ. Along the same lines, the real flow Q will vary as a function of time, and will cause divergent behaviour in phases of low flow (such as during late diastolic phases, where Q→0). On the contrary, in the case of an introduced virtual field, the key benefit is that mass conservation between Γᵢ and Γₒ can be enforced, and the virtual Q will no longer be affected by low real flow magnitudes. This difference becomes particularly apparent in the utilized in-silico model where vWERP-t shows maintained accuracy, whereas WERP-t and the other real work-energy approaches diverge following the complex model anatomy and varying real flow magnitudes.

However, the differences observed for the steady-state stenotic flow phantoms is less intuitive. In principle, for a steady-state single-vessel flow, there should not be any major differences between WERP-t and the virtual equivalent of vWERP-t.

Regardless, the inventors observed that WERP-t did generate systematic underestimations, whereas vWERP-t maintained accurate method output in the in-vitro cohort.

Hence, regardless of the turbulent nature of the acquired field, divergence-free behaviour is guaranteed in vWERP-t, ensuring that fundamental constraints are withheld in the evaluated virtual work-energy setup. Along the same lines, the apparent difference in energy component weighting between virtual and real work-energy approaches also have clinical implications. As observed, even in cases of dominant real turbulent energy dissipation, the use of a virtual field seem to enhance advective energy weighting compared to its real work-energy equivalent.

Consequently, vWERP-t will rely on accurate mean field quantities to a higher extent than WERP-t, where focus will rather be on maintained covariance capturing.

Although various techniques have been described in terms of certain aspects and embodiments, each can be combined to provide further aspects and embodiments. In addition, certain features shown in the context of one aspect and/or embodiment can be incorporated into other aspects and/or embodiments as well.

### Examples

Example 1 is a method of determining a pressure difference across a hollow region arising from fluid flow within the hollow region, comprising: obtaining three-dimensional time-dependent fluid flow data; processing the three-dimensional time-dependent fluid flow data to derive mean field flow data and flow covariance data corresponding to the mean field flow data; processing the mean field flow data to define a fluid flow domain (Ω, Ω_{ROI}) over which the pressure difference is to be determined; de-noising the flow covariance data; computing a arbitrary velocity field (w), wherein the arbitrary velocity field is a solenoidal field with zero velocity on a lateral wall region (Γ_{w}) of the fluid flow domain (Ω_{ROI}); processing the de-noised flow covariance data, the mean field flow data and the arbitrary velocity field (w) to determine: (i) a flow rate (Q) as a function of the arbitrary velocity field (w); (ii) a virtual kinetic energy (Kₑ) of the fluid flow; (ii) a virtual advective energy rate (Aₑ) of the fluid flow; (iii) a virtual viscous dissipation rate (Vₑ) pertaining to the fluid flow; and (iv) a virtual turbulent energy dissipation (Tₑ) of the fluid flow; and calculating the pressure difference in dependence on all of the flow rate (Q), kinetic energy (Kₑ), advective energy rate (Aₑ), viscous dissipation rate (Vₑ) and turbulent energy dissipation (Tₑ).

Example 2 is a method according to Example 1 wherein the arbitrary velocity field (w) is computed using a Finite Difference Method with staggered grid approach.

Example 3 is a method according to Example 1 further comprising using a kernel-based k-order three-dimensional polynomial fitting to de-noise the mean field flow data.

Example 4 is a method according to Example 1 further comprising calibrating and correcting the obtained time-dependent three dimensional fluid flow data to remove potentially erroneous data relating to at least eddy currents, field inhomogeneities, and aliasing.

Example 5 method according to Example 1, wherein the hollow region is part of a cardiovascular structure and the three-dimensional time-dependent fluid flow data is obtained over a plurality of cardiac cycles with a period T.

Example 6 is a method according to Example 1 wherein processing the mean field flow data to define a fluid flow domain (Ω_{ROI}) comprises: creating a segmented fluid flow domain; selecting an inlet plane (Γ_{I,ROI}) and an outlet plane (Γ_{O,ROI}) for the fluid flow domain, wherein a cross-section of the inlet plane (Γ_{I,ROI}) is perpendicular to the fluid flow domain and a cross-section of the outlet plane (Γ_{O,ROI}) is perpendicular to the fluid flow domain; and labelling of the segmented fluid domain such that the segmented fluid flow domain is adapted for assignment of boundary conditions for computation of the arbitrary velocity field (w).

Example 7 is a method according to Example 6, wherein the three dimensional time-dependent fluid flow data comprises velocity magnitude images relating to the velocity field of the fluid flow and wherein creating the segmented fluid flow domain comprises using a binary image mask to isolate the fluid flow domain by thresholding based on the velocity magnitude images.

Example 8 is a method according to Example 7, further comprising subsampling the segmented fluid flow domain prior to the computation of the arbitrary velocity field.

Example 9 is a method according to Example 8, wherein the segmented fluid flow domain is a voxelised fluid flow domain and wherein subsampling the segmented fluid flow domain comprises converting each image voxel of the fluid domain into a plurality of uniformly distributed subvoxels and optionally wherein the subsampling comprises subdividing each image voxel into eight uniformly distributed subvoxels.

1. A method of estimating pressure difference across a hollow region arising from fluid flow within the hollow region, the method comprising:
acquiring a three-dimensional time-dependent image of the fluid flow;
processing the acquired image to derive an expression of the mechanical behaviour of the fluid flow, wherein the expression comprises a component relating to stochastic flow fluctuations in the fluid flow;
processing at least part of the derived expression of the fluid flow mechanical behaviour to define a fluid flow domain;
computing an arbitrary, divergence-free velocity field (w) with null values on a lateral wall (Γ_{w}) of the fluid flow domain (Ω, Ω_{ROI});
processing components of the derived expression in combination with the arbitrary flow field to derive a work-energy expression for the fluid flow; and
estimating the said pressure difference using elements of the derived work-energy expression.

2. A method according to claim 1, wherein processing the acquired image to derive the expression of the mechanical behaviour of the fluid flow comprises deriving mean field flow data and flow covariance data corresponding to the mean field flow data.

3. A method according to claim 2, wherein the mean field flow data comprises mean velocity field data (V) and mean pressure field (P), where
V= [symbol] [v]P; and
P= [symbol] [p]; and
wherein v is the velocity field data of the fluid flow and p is the pressure field data of the fluid flow and E is the linear expected value operator.

4. A method according to claim 3, wherein processing at least part of the derived expression of the fluid flow mechanical behaviour to define the fluid flow domain comprises processing the mean field flow data to define the fluid flow domain over which the pressure difference is to be determined.

5. A method according to claim 3, wherein processing components of the derived expression in combination with the arbitrary flow field to derive the work-energy expression for the fluid flow comprises: de-noising the flow covariance data; processing the de-noised flow covariance data, the mean field flow data and the arbitrary velocity field (w) to determine:
(i) a flow rate (Q) as a function of the arbitrary velocity field (w);
(ii) a virtual kinetic energy (Kₑ) of the fluid flow;
(ii) a virtual advective energy rate (Aₑ) of the fluid flow;
(iii) a virtual viscous dissipation rate (Vₑ) pertaining to the fluid flow; and
(iv) a virtual turbulent energy dissipation (Tₑ) of the fluid flow.

6. A method according to claim 5, wherein the pressure difference is given by:
$$Δ p = - \frac{1}{Q} ( \frac{\partial }{\partial t} {K}_{e} + {A}_{e} + {V}_{e} + {T}_{e} ) .$$

7. A method according to claim 5, wherein:
(i) the flow rate (Q) is dependent on a surface integral of the arbitrary velocity field (w) across either an inlet (Γᵢ) or an outlet (Γₒ) plane of the fluid flow domain, or wherein the flow rate (Q) is computed as a weighted average of aforementioned surface integrals; and/or
(ii) the virtual turbulent energy dissipation term (Tₑ) is dependent on the flow covariance data, the arbitrary velocity field (w) and the fluid density (p); and/or
(iii) the virtual kinetic energy rate (Kₑ) is dependent on the arbitrary velocity field (w), the velocity field data (v) of the fluid flow, and the fluid density (p); and/or
(iv) the virtual advected energy rate (Aₑ) is dependent on a sum of the surface integrals of the arbitrary velocity field (w) and the velocity field data (v) of the fluid flow, or data derived therefrom, across inlet and outlet planes of the fluid flow domain and the fluid density (p); and/or
(v) the virtual viscous dissipation rate (Vₑ) is dependent on the virtual velocity field (w) and the velocity field data (v) of the fluid flow and dynamic viscosity (μ) of the fluid.

8. A method according to claim 1, wherein the arbitrary velocity field (w) is computed as a solution to:
$${∇}^{2} w + ∇ λ = 0$$
$$∇ \cdot w = 0$$
$$w = \{ \begin{matrix}- n ( 1 - {( \frac{r}{R} )}^{2} ) , & {Γ}_{i} \\ 0 , & {Γ}_{w}\end{matrix}$$
wherein λ is the virtual pressure field corresponding to the arbitrary velocity field (w) and n is the normal vector on an inlet plane Γᵢ of a boundary Γ of the flow domain Ω, wherein a parabolic inflow is defined at Γᵢ in a direction of the normal n, at a radial position r with a perimeter radius R.

9. A method according to claim 5, wherein de-noising the flow covariance data comprises removing non-physical negative diagonal entries from the flow covariance data.

10. A method according to claim 4, wherein processing the mean field flow data to define the fluid flow domain (Ω_{ROI}) comprises:
creating a segmented fluid flow domain;
selecting an inlet plane (Γ_{I,ROI}) and an outlet plane (Γ_{O,ROI}) for the fluid flow domain, wherein a cross-section of the inlet plane (Γ_{I,ROI}) is perpendicular to the fluid flow domain and a cross-section of the outlet plane (Γ_{O,ROI}) is perpendicular to the fluid flow domain; and
labelling of the segmented fluid domain such that the segmented fluid flow domain is adapted for assignment of boundary conditions for computation of the arbitrary velocity field (w).

11. A method according to claim 5, wherein the fluid flow domain (Ω_{ROI}) is a spatiotemporally discretised fluid flow domain, wherein:
$${K}_{e} ( v , w ) = ρ dV \text{?} ( v ( i , j , k ) \cdot w ( i , j , k ) )$$
$${A}_{e} ( v , w ) = ρ dV \text{?} ( [ v ( i , j , k ) \cdot G ( v ) ( i , j , k ) ] \cdot w ( i , j , k ) )$$
$${V}_{e} ( v , w ) = μ dV \text{?}$$
$${T}_{e} ( v , w ) = - pdV \text{?} ( Cov [ v , v ] ( i , j , k ) \text{:} G ( w ) ( i , j , k ) )$$
$$Q ( w ) = dS \text{?} ( w ( i , j ) \cdot N ( i , j ) ) , \\ \text{?} \text{indicates text missing or illegible when filed}$$
wherein the inlet plane (Γ_{I,ROI}) and the outlet plane (Γ_{O,ROI}) of the fluid domain (Ω_{ROI}) have corresponding normal vectors (N) and where (i, j, k) are voxel indices; dS=Π [symbol] ²Δxᵢ and dV=Π [symbol] ³Δxᵢ are the pixel area and voxel volume, respectively, both based on the voxel length Δxᵢ in each spatial dimension; and G(⋅) is the discretized gradient operator, defined as a spatial central-difference operator with one-directional derivatives employed at boundaries of the fluid flow domain.

12. A method according to claim 11, wherein the pressure difference is given by:
$$Δ \text{?} = - \frac{1}{Q ( w )} ( \frac{\partial }{\partial t} {K}_{e} ( \text{?} , w ) + {A}_{e} ( \text{?} , w ) + {V}_{e} ( \text{?} , w ) + {T}_{e} ( \text{?} , w ) , \\ \text{?} \text{indicates text missing or illegible when filed}$$
with
$$\text{?}$$
$$\text{?} \text{indicates text missing or illegible when filed}$$
derived as the mean of vₜ and vₜ₊₁.

13. A method according to claim 10 further comprising subsampling the segmented fluid flow domain prior to the computation of the arbitrary velocity field.

14. A method according to claim 1, wherein the hollow region is part of an in-vivo cardiovascular structure and the three-dimensional time-dependent fluid flow data is obtained over a plurality of cardiac cycles with a period T.

15. A method according to claim 1, wherein the fluid is blood and the hollow region is an in-vivo blood vessel.

16. A method according to claim 1, wherein the method is a computer-implemented method.

17. A system comprising:
a unit configured to receive imaged fluid flow data across a hollow region;
a non-transitory computer-readable medium storing a program causing a computer to execute a process on the imaged fluid flow data for estimating a pressure difference due to the fluid flow across the hollow region, wherein the program is further adapted to cause the computer to estimate a virtual work-energy contribution to the pressure difference due to stochastic fluctuations in the fluid flow;
a processor configured to execute the program stored on the non-transitory computer-readable medium;
a unit configured to store the estimated pressure difference data.

18. The system according to claim 17 further comprising a unit configured to image fluid flow data across a hollow region.

19. The system according to claim 18, wherein the unit is a 4D PC-MRI scanner.

20. A non-transitory computer-readable medium storing a program causing a computer to execute a process on imaged fluid flow data, the program comprising instructions to:
acquire a three-dimensional time-dependent image of the fluid flow;
process the acquired image to derive an expression of the mechanical behaviour of the fluid flow, wherein the expression comprises a component relating to stochastic flow fluctuations in the fluid flow;
process at least part of the derived expression of the fluid flow mechanical behaviour to define a fluid flow domain;
compute an arbitrary, divergence-free velocity field (w) with null values on a lateral wall (Γ_{w}) of the fluid flow domain (Ω, Ω_{ROI});
process components of the derived expression in combination with the arbitrary flow field to derive a work-energy expression for the fluid flow; and
estimate the said pressure difference using elements of the derived work-energy expression.
